(19) United States
(12) Patent Application Publication
(10) Pub. No.: US 2026/0066073 A1
(43) Pub. Date: Mar. 5, 2026

(54) GENERATIVE AI-BASED SYSTEMS AND METHODS FOR AUTOMATICALLY GENERATING PATIENT-SPECIFIC COMMUNICATIONS

(71) Applicant: KONINKLIJKE PHILIPS N.V., EINDHOVEN (NL)

(72) Inventors: FALK UHLEMANN, NORDERSTEDT (DE); MICHAEL GÜNTER HELLE, HAMBURG (DE); DIRK SCHAEFER, HAMBURG (DE); THOMAS ERIK AMTHOR, HAMBURG (DE); STEFFEN RENISCH, HAMBURG (DE)

(21) Appl. No.: 19/302,607

(22) Filed: Aug. 18, 2025

Related U.S. Application Data
(60) Provisional application No. 63/687,845, filed on Aug. 28, 2024.

(51) Int. Cl. G16H 15/00

(52) U.S. Cl. CPC G16H 15/00

(57) ABSTRACT

A computer-implemented system for automatically generating patient-specific medical reports includes a data collection module configured to obtain patient-specific information, including patient-specific diagnostic information and communication-related patient-specific meta information. The system further includes an artificial intelligence (AI) module configured to process the obtained patient-specific information to generate a patient-specific report in a format tailored to the patient's language, education, and expectation level, wherein the AI module includes a pre-trained language model trained using medical terms, texts, reports, and related images for different age, language, and educational level groups. The system further includes an output module configured to output the generated report.

### CROSS REFERENCE TO RELATED APPLICATIONS

This application claims the benefit of U.S. Provisional Patent Application No. 63/687,845 filed Aug. 28, 2024. This application is hereby incorporated by reference herein.

### FIELD

The inventive concepts generally related to healthcare, and in particular, to Generative AI-based systems and methods for automatically generating patient-specific communications.

### BACKGROUND

In the field of healthcare, communication between medical professionals and patients is of utmost relevance. This communication often involves the sharing of diagnostic information, which can be complex and difficult for patients to comprehend. This information typically includes laboratory results, radiologic findings, and other patient-specific diagnostic data. The complexity of this information is further amplified by the continuous advancements in diagnostic technologies, such as biomarker tests and multimodality imaging.

The communication of this information is often facilitated through medical reports. These reports are typically written in a language that is comprehensible to medical professionals but can be challenging for patients to understand due to the use of medical terms and jargon. The language, terminology, and level of detail used in these reports are not typically tailored to the patient's age, native language, or educational background, which can further hinder the patient's understanding of their medical condition and the recommended treatment plan.

In addition to the textual information, medical reports may also include images, such as radiologic images, to provide a visual representation of the patient's medical condition. However, these images are often difficult for patients to interpret without the aid of a medical professional. Furthermore, these images are typically not personalized or adapted to the patient's specific situation or diagnosis, which can make it challenging for patients to understand the implications of the images in the context of their personal health.

Separately, the process of creating these reports is often time-consuming. Doctors typically have to manually extract the relevant information from the various sources and then communicate this information to the patient in a way that they can understand. This process can be stressful for both the doctor and the patient and can potentially lead to miscommunication, which in turn can affect the choice and delivery of therapy.

### SUMMARY

According to an aspect of the inventive concepts, a computer-implemented system is provided for automatically generating patient-specific medical reports. The system comprises a processor configured to: obtain patient-specific information, including patient-specific diagnostic information and communication-related patient-specific meta information; process the obtained patient-specific information using a pre-trained language model to generate a patient-specific report in a format tailored to the patient's language, education, and expectation level, wherein the pre-trained language model is trained using medical terms and medical data for different age, language, and educational level groups; and output the generated report.

The processor may be configured to obtain the patient-specific diagnostic information from a variety of sources, including at least one of laboratory results, radiologic findings, or images. The processor may be further configured to use the collected patient-specific diagnostic information to generate the patient-specific report to include a detailed explanation of the patient's medical condition, potential consequences, and personalized instructions. The processor may be further configured to generate the patient-specific report to include text with adapted terminology and an extendable full-length detailed report. The processor may be further configured to create a medical image of the obtained patient-specific diagnostic information showing a radiologic finding and include the medical image in the patient-specific report. The processor may be further configured to augment a textual explanation provided in the patient-specific report with the medical image. The processor may be further configured to refine the pre-trained language model using patient feedback responses on the generated patient-specific report. The system may further comprise a second processor configured to train the pre-trained language model using the medical terms and medical data for different age, language, and educational level groups.

According to another aspect of the inventive concepts, a computer-implemented method is provided for automatically generating patient-specific medical reports. The method comprises: obtaining patient-specific information, including patient-specific diagnostic information and communication-related patient-specific meta information; processing the obtained patient-specific information using a pre-trained language model to generate a patient-specific report in a format tailored to the patient's language, education, and expectation level, wherein the pre-trained language model is trained using medical terms and medical data for different age, language, and educational level groups; and outputting the generated report.

According to another aspect of the inventive concepts, a computer-implemented method is provided a non-transitory computer-readable medium having stored thereon instructions which, when executed by a processor, cause the processor to obtain patient-specific information, including patient-specific diagnostic information and communication-related patient-specific meta information; process the obtained patient-specific information using a pre-trained language model to generate a patient-specific report in a format tailored to the patient's language, education, and expectation level, wherein the pre-trained language model is trained using medical terms and medical data for different age, language, and educational level groups; and output the generated report.

According to another aspect of the inventive concepts, a computer-implemented system is provided for automatically generating patient-specific medical reports. The system comprises a data collection module configured to collect patient-specific diagnostic information and communication-related patient-specific meta information. The system further includes a generative artificial intelligence (AI) module configured to process the collected information and generate a patient-specific report, wherein the generative AI module includes pre-trained language models trained using medical terms, texts, reports, and related images for different age, language, and educational level groups. The system still further includes an output module configured to present the generated report to the patient in a format tailored to the patient's language, education, and expectation level.

The data collection module may further be configured to collect patient-specific diagnostic information from a variety of sources including laboratory results, radiologic findings, and a full set of images.

The generative AI module may further be configured to use the collected patient-specific diagnostic information to generate a patient-specific report that includes a detailed explanation of the patient's medical condition, potential consequences, and personalized instructions.

The output module may further be configured to present the generated report to the patient in a format that is tailored to the patient's language, education, and expectation level, and includes text with adapted terminology and an extendable full-length detailed report.

The generative AI module may further be configured to create a healthy and more severe version of a medical image showing a radiologic finding, and to include these images in the patient-specific report to allow easy comparison of the healthy/more severe situation to the patient's situation.

The generative AI module may further be configured to highlight relevant features in the patient-specific and reference images to augment the textual explanation provided in the patient-specific report.

The system may further include a feedback module configured for continuous improvement of the generative artificial intelligence (AI) module, wherein the feedback module is configured to collect and apply patient feedback responses to the generative artificial intelligence (AI) module.

The system may further include a training module configured to train the language models of the generative AI module using the medical terms, texts, reports, and related images for different age, language, and educational level groups

According to another aspect of the inventive concepts, a computer-implemented method is provided for automatically generating patient-specific medical reports. The method includes collecting patient-specific diagnostic information and communication-related patient-specific meta information, and processing the collected information using a generative artificial intelligence (AI) module, wherein the generative AI module includes pre-trained language models trained using medical terms, texts, reports, and related images for different age, language, and educational level groups. The method further includes generating a patient-specific report based on the processed information, and presenting the generated report to the patient in a format tailored to the patient's language, education, and expectation level.

The patient-specific diagnostic information may include laboratory results, radiologic findings, and a full set of images.

The generative AI module may further be configured to use the collected patient-specific diagnostic information to generate a patient-specific report that includes a detailed explanation of the patient's medical condition, potential consequences, and personalized instructions.

The generated report may be presented to the patient in a format that is tailored to the patient's language, education, and expectation level, and includes text with adapted terminology and an extendable full-length detailed report.

The generative AI module may further be configured to create a healthy and more severe version of a medical image showing a radiologic finding, and to include this image in the patient-specific report.

The generative AI module may further be configured to highlight relevant features in the patient-specific and reference images to augment the textual explanation provided in the patient-specific report.

According to still another aspect of the inventive concepts, a non-transient computer-readable medium having stored thereon instructions that, when executed by a processor, cause a system to collect patient-specific diagnostic information and communication-related patient-specific meta information, and process the collected information using a generative artificial intelligence (AI) module, wherein the generative AI module includes pre-trained language models trained using medical terms, texts, reports, and related images for different age, language, and educational level groups. The instructions further cause the system to generate a patient-specific report based on the processed information, and present the generated report to the patient in a format tailored to the patient's language, education, and expectation level. The patient-specific diagnostic information may include laboratory results, radiologic findings, and a full set of images.

### BRIEF DESCRIPTION OF THE DRAWINGS

The above and other aspects and features of the inventive concepts will become readily apparent from the detailed description that follows, with reference to the accompanying drawings, in which:

FIG. 1 schematically depicts an example computing environment in which selected aspects of embodiments of the inventive concepts may be implemented:

FIG. 2 is a flow diagram for reference in describing the automatic generation of patient-specific communications in accordance with embodiments of the inventive concepts;

FIG. 3 is a system block diagram in describing the automatic generation of patient-specific communications in accordance with embodiments of the inventive concepts; and

FIGS. 4 and 5 are comparative patient communications for reference in describing an example of a patient-specific communication automatically generated in accordance with embodiments of the inventive concepts.

### DETAILED DESCRIPTION

In the following detailed description, for purposes of explanation and not limitation, representative embodiments disclosing specific details are set forth in order to provide a thorough understanding of the present teachings. However, it will be apparent to one having ordinary skill in the art having had the benefit of the present disclosure that other embodiments according to the present teachings that depart from the specific details disclosed herein remain within the scope of the appended claims. Moreover, descriptions of well-known apparatuses and methods may be omitted to avoid obscuring the description of the example embodiments. Such methods and apparatuses are clearly within the scope of the present teachings. Further, throughout the drawings, like reference numbers refer to the same or similar elements.

The terminology used herein is for purposes of describing particular embodiments only, and is not intended to be limiting. The defined terms are in addition to the technical and scientific meanings of the defined terms as commonly understood and accepted in the technical field of the present teachings. As used in the specification and appended claims, the terms ‘a’, ‘an’ and ‘the’ include both singular and plural referents, unless the context clearly dictates otherwise. Thus, for example, ‘a device’ includes one device and plural devices. Further, for example, when one element is described as being “connected to” another element, the one element may be directly connected to the other element, or indirectly connected to the other element in an operative manner.

Separately, as is traditional in the field of the inventive concepts, example embodiments may be described, and illustrated in the drawings, in terms of functional blocks, units and/or modules. Those skilled in the art will appreciate that these blocks, units and/or modules are physically implemented by electronic (or optical) circuits such as logic circuits, discrete components, microprocessors, hard-wired circuits, memory elements, wiring connections, and the like, which may be formed using semiconductor-based fabrication techniques or other manufacturing technologies. In the case of the blocks, in the absence of an indication to the contrary, the units and/or modules being implemented by microprocessors or similar may be programmed using software (e.g., microcode) to perform various functions discussed herein and may optionally be driven by firmware and/or software. Alternatively, each block, unit and/or module may be implemented by dedicated hardware, or as a combination of dedicated hardware to perform some functions and a processor (e.g., one or more programmed microprocessors and associated circuitry) to perform other functions. Also, each block, unit and/or module of the example embodiments may be physically separated into two or more interacting and discrete blocks, units and/or modules without departing from the scope of the example embodiments. Conversely, the blocks, units and/or modules of the example embodiments may be physically combined into more complex blocks, units and/or modules without departing from the scope of the example embodiments.

The inventive concepts are directed to a system for the automatic generation of patient-specific communications, such as reports, based on detailed patient-specific diagnostic information and communication-related patient-specific meta information. The system utilizes a Generative AI module with pre-trained language models, specifically trained using medical terms, texts, reports, and related images for different age, language, and educational level groups. The system also employs generative AI or inpainting to create “healthy” and/or “more severe” versions of a medical image showing a radiologic finding.

In some embodiments, the system may include an additional initial training phase. During this phase, a large training data set may be collected and used or a previously generated large training data set may be used. This data set may include pre-existing or historical data including medical reports covering relevant diseases or cases, general texts containing medical terms or descriptions at different language levels, sets of annotated image data that may be tied to medical report texts, and a mapping between target groups and contexts. The mapping may include associations between language levels and the size of the generated report, as well as suitable inference steering phrases, also known as “prompts”.

The generative AI model may trained or built based on this large training data set. This model is multimodal, meaning it is capable of processing and generating both language and images. The training of the model may be performed using known methods of generative AI model creation.

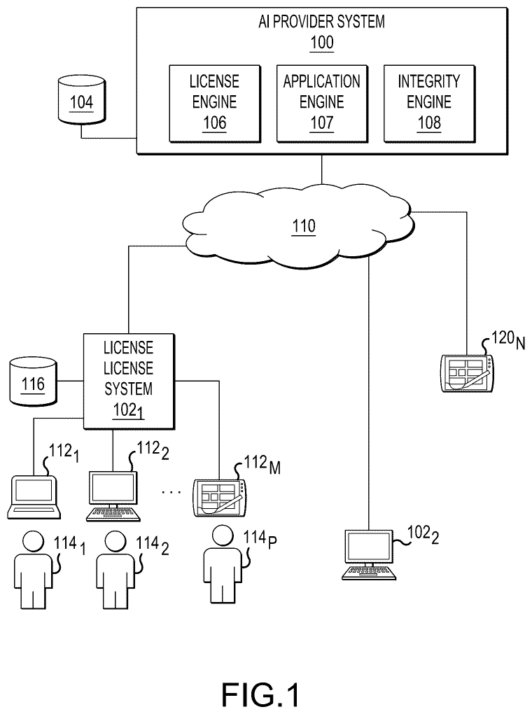

FIG. 1 is a schematic diagram of an operating environment in which aspects of the inventive concepts may be implemented.

Referring to FIG. 1, an AI provider system 100 may include one or more computing devices and/or servers (e.g., blade servers) that are operated by an entity such as a business, government entity, individual, non-profit, organization, etc., to provide AI services to others. In various embodiments, the AI provider system 100 may be communicatively coupled with one or more remote computing systems 102_{1-N} over one or more wired and/or wireless computing networks 110 (e.g., the Internet). In some implementations, AI provider system may be implemented across multiple computing devices forming what may be referred to as a “cloud” infrastructure or “the cloud.”

In various implementations, AI provider system 100 may provide, to one or more individuals (“users”), access to one or more machine learning (“ML”) models. In some embodiments, AI provider system 100 may provide various levels of ML model access to different individuals and/or entities, depending on credentials provided by and/or on behalf of the individuals and/or entities.

In FIG. 1, a first remote computing system 102; may take the form of, for instance, a healthcare or hospital computing system/network that includes one or more computing devices, servers, instruments, smart appliances, networked medical devices, etc. The first remote computing system 102₁ may further include, for example, various healthcare-related computing subsystems (not depicted), such as a hospital information system (“HIS”), an electronic health records (“EHR”) database, and so forth. In various implementations, a plurality of client devices 112_{1-M} operated by a plurality of users 1141-p (e.g., medical personnel) may connect to computing system 102₁, e.g., over one or more networks (not depicted, could include the Internet). In addition to or instead of servicing systems of multiple computing devices, AI provider system 100 may service individual computing devices, such as 102₂ and 102_{N}.

In the case where the first remote computing system 102₁ is a healthcare or hospital computing system, the embodiments described later herein may, for example, be implemented by the combination of the computer resources of the first remote computing system 102₁ and the AI provider system 100. However, the inventive concepts are not limited in this manner. For example, rather than cloud-based, the AI functionality utilized in the embodiments may be configured locally within the first remote computing system 102₁.

In the example of FIG. 1, one or more ML models may be stored by AI provider system 100 in a ML model database 104. These ML models may take various forms, such as deep learning neural networks, recurrent neural networks (“RNNs”), convolutional neural networks (“CNNs”), support vector machines, decision trees, reinforcement learning models, adversarial generative networks (“GANs”), and so forth. The AI provider system 100 may make these ML models available to remote computing systems 102 in various ways. In some implementations, remote computing systems 102 may download copies of ML models from the database 104 and store them locally. e.g., in their own databases (e.g., 116). Additionally or alternatively, in some implementations, the AI provider service 100 may retain the ML models exclusively in database 104, and may apply data received from remote the computing systems 102 across the ML models on demand. In some such implementations, the AI provider system 100 and/or the remote computing system 102 may encrypt the exchanged data (e.g., input to the ML model, output generated from the ML model, etc.).

In some implementations, AI provider system 100 may include a license engine 106, an application engine 107, and/or an integrity engine 108. These engines may be implemented using any combination of software and hardware, and may be implemented on a single computing device or across multiple computing devices (e.g., as “cloud-based components”). In other embodiments, one or more of engines 106-108 may be combined and/or omitted, or implemented at least in part on another computing system, such as on remote computing system 102₁.

License engine 106 may be configured to examine credentials provided by or on behalf of individual users and/or entities to determine which ML models the individual users/entities should have access, what level of access the individual users/entities should have to each ML model, how long and/or for how many distinct uses each individual user/entity should have access to particular ML models, etc. Put another way, license engine 106 may be configured to authenticate users and/or entities to use various ML models, whether stored locally by AI provider service 100 in database 104 and/or stored remotely at one or more remote computing systems 102 (e.g., in database 116).

Application engine 107 may be configured to apply input across the one or more ML models stored in database 104 to generate output. As mentioned previously, in some embodiments, AI provider service 100 may retain ML models locally in database 104, and may, by way of application engine 107, receive input from remote computing systems 102 that is to be applied across those ML models by application engine 107. For example, in some implementations, a remote computing system 102 may provide input data (e.g., digital images, waveforms, text, etc.) to AI provider service 100. Application engine 107 may apply this input data across one or more ML models (e.g., for which the entity/user operating the remote computing system 102 is licensed, selects, etc.) to generate output. Data indicative of the output, and/or the output itself, may be returned to the remote computing system 102. In other embodiments, however, one or more remote computing systems 102, such as entity computing system 102₁, may download the ML models it will be using and store them locally, e.g., in database 116. In some such embodiments, the remote computing system 102 may have its own application engine (not depicted) that applies data across the locally-stored ML model.

Integrity engine 108 may be configured to examine various aspects of ML models stored locally to AI provider system 100 (e.g., in database 104) and/or remotely, e.g., in database 116, to determine whether and/or how those ML models may have been compromised. For example, a malicious party may gain access to a ML model stored in database 116 and may alter one or more aspects of the ML model, such as altering or deleting one or more parameters or weights in various layers. Alternatively, a licensed entity may attempt to make changes to its locally stored model when it is not licensed to do so. In either case, integrity engine 108 may be configured to apply various techniques described herein, or cause these techniques to be applied at one or more remote computing systems 102, in order to verify the integrity of a ML model and/or to take appropriate remedial action when it determines that a ML model has been compromised. In some embodiments, integrity engine 108 may verify the integrity of a ML model by applying a digital key as input across the ML model to generate output, which is then verified by integrity engine 108 as described herein.

In some implementations in which a ML model is stored remotely from AI provider system 100, e.g., in database 116, one or more client devices 112_{1-M} and/or entity computing system 102₁ may host a software application that is operable by end users 114 to make use of the ML model. In some implementations, this software application may be provided (e.g., in an app store) and maintained by AI provider system 100. In some such embodiments, AI provider system 100, e.g., by way of integrity engine 108, may retain the right to cause the remotely-executing software application to periodically perform techniques described herein, such as techniques employed by integrity engine 108.

For example, in some embodiments, when a particular user 114 (e.g., a nurse) operates a client device 112 to interact with the software application, the nurse may log into the client device 112 with one or more credentials. These credentials may authenticate the nurse to utilize the software application to apply data across one or more ML models. The nurse may not be made explicitly aware that he or she will be accessing a ML model. Rather, the nurse may simply interact with a graphical user interface (“GUT”) or other input component. In some embodiments, the nurse's credentials may restrict the nurse's access to some models, while denying the nurse access to other models (which, if the nurse attempted to use functionality that relied on restricted models, might provide audio or visual output such as “You are not authorized to perform this operation”) Additionally or alternatively, in some embodiments, the nurse's credentials may restrict what is ultimately output from the model, as will be described in more detail below.

The inventive concepts are direct to the automatic generation of patient specific communications in a hospital or healthcare environment such the example described above in connection with FIG. 1.

The embodiments that will described below overcome a number of drawbacks of conventional techniques. Traditionally, clinical reports, specific consequences and personalized instructions are often complex and difficult to understand, even though is important that the reports be understood by patients (e.g. to improve adherence to prescribed medication). Further, creation and delivery of patient-specific communication (e.g. explanation or letter in tailored language/terms) takes significant time and cannot practically be provided in all cases due to limited patient/doctor availability. Separately, the complexity of diagnostic information is steadily increasing due to technical advances (e.g. biomarker tests, multimodality imaging), but only relevant information should be communicated by default. These issues are currently addressed by doctors manually/case-by-case extracting and communicating the essential information to the patient in a way that (hopefully) can be understood and recalled by the patient. This induces stress on the doctor's and patient's side and hampers an efficient execution of medical procedures. Due to miscommunication this can even lead to a suboptimal choice and delivery of therapy (e.g. due to lacking patient adherence, missing patient feedback/additional information to diagnostic findings).

To overcome these drawbacks, in embodiments of the inventive concepts, the communications (or reports) are generated automatically based on detailed patient-specific diagnostic information (e.g. laboratory results, radiologic findings and full set of images), as well as communication related patient-specific meta information (e.g. age, native language, educational background, history/records of previous conversations, electronic health records (HER), and so on.)

In embodiments of the inventive concepts, the communications are generated using Generative AI with pre-trained language models that are built using medical terms/texts/reports and related images for different age/language/educational level groups. In addition, Generative AI inpainting is leveraged to create a “healthy” and/or “more severe” versions of a medical image showing and allowing easy comparison of a radiologic finding.

As a result, patient-specific information is generated that is tailored to the language, education and expectation level of the patient. For example, the meta data of one patient may indicated that the text with adapted terminology should be relatively short and simple, while for others extended full-length detailed report and interactive version is appropriate. Likewise, patient-specific reference images (optionally at multiple levels/degrees of detail may be presented (e.g. initial personal communication versus detailed “take home” information) including highlighting of relevant features in patient and reference images to augment textual explanations.

The AI-generated “healthy” and/or “severe” version of the same view helps an untrained person (patient) understand what exactly has been found in the image and how the image would have looked like in a healthy state and/or a more severe state to ease understanding or illustrate possible progression including automatically created links to patient image data including highlighted relevant structures. For example, illustrating healthy and severe states (alongside the patient's actual images) and annotations using AI-based inpainting/segmentation/simplification in actual image data of the respective patient to allow easy alignment and understanding of differences/progression by the patient, e.g. compare current state with healthy tissue or healthy organ which is AI-generated.

The reports may include an outlook on next steps to be discussed with the physician, e.g. treatment options, medication & medication plan, and hands-on advice what the patient can do until the next appointment (for an advanced diagnosis or beginning of treatment), including medication plan.

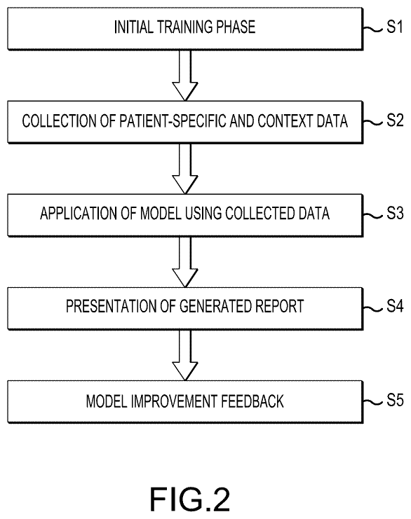

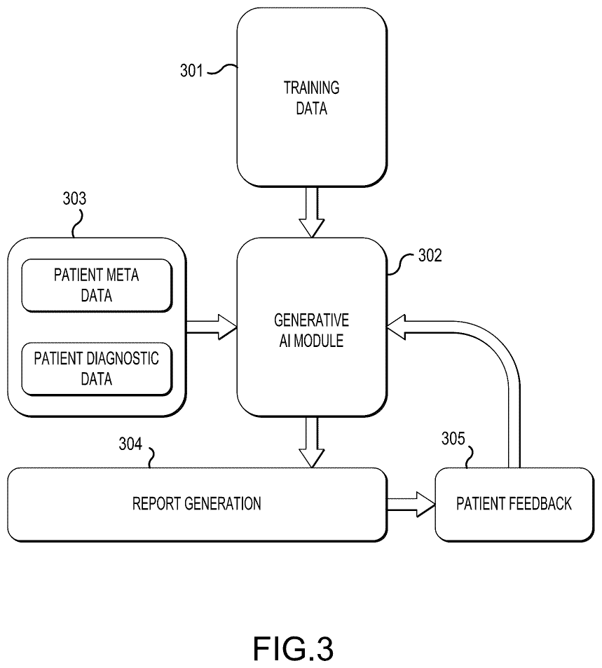

FIG. 2 is a flow diagram for reference in describing an example of a radiographic report for a patient, and FIG. 3 is a system diagram for generating a radiographic report for a patient according to embodiments of the inventive concepts. As shown, the system is made up of a training data module 301, a Generative AI module 302, a data collection module 303, a report generation (or output) module 304, and a patient feedback module 305. The following description uses the generation of a radiographic report for a patient as example, but the inventive concepts are not limited in this manner.

Referring collectively to FIGS. 2 and 3, an initial training phase (S1) of the Generative AI module 302 is carried out using the training data module 301 and known methods of generative AI model creation. Training is based on a large training data set containing:
Medical reports covering relevant diseases/cases
General texts containing medical terms/descriptions at different language levels as tags
Sets of (2D/3D) annotated image data (possibly tied to medical report texts) serving as reference images during report generation phase
Mapping between target groups (e.g. language levels) and contexts (e.g. size of generated report) and suitable inference steering phrases (“prompts”)

As a result, a multimodal (language and images) generative model is trained and created.

Next, patient-specific and context data is collected (S2) using the data collection module 303. This data is generally of two types, i.e., patient diagnostic data and patient meta data. The data may be collected (obtained) from one or more of medical records, reports, images, structured or unstructured input by a user or clinician, or any other document or format of patient information without limitation.

The patient diagnostic data includes central patient-specific diagnostic findings (e.g. “bleeding in left hemisphere visible at location . . . ”). If the data is not made directly available from the radiologist, is could also be extracted by known/available techniques of textual abstract generation. The patient's imaging data is also collected to enable multimodal report generation.

Patient meta data includes patient-specific information related to communication level (e.g. age, native language, impairments, educational level). The is the data used to map between target groups and contexts as mentioned above in the training phase.

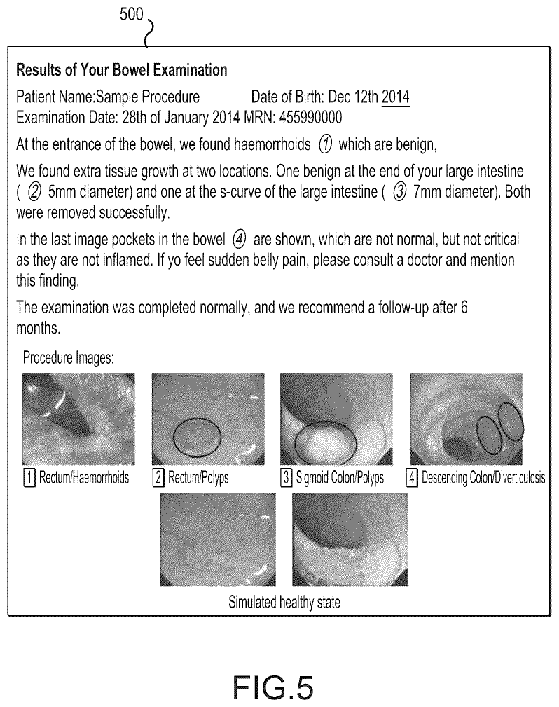

Next, the collected data is applied (S3) to the pre-trained models of the Generative AI model 302. Specifically, using the mapped “communication level” phrases and the central diagnostic findings, model inference is started. Employing the annotated (reference) image data and using AI-based inpainting, reference images (e.g. with a severe and normal state of the anatomy) using the patient's images as “background” can be created and displayed next to the unmodified corresponding patient image. An example of this will be described below in connection with FIG. 5.

Next, using the report generation module 304, the report is generated and communicated to the patient (S4). The generated report is optionally at different language/detail levels for different “channels” (e.g. oral, written or interactive web-based).

In embodiments of the inventive concepts, the patient feedback module 305 is provided as a mechanism for model improvement (S5). That is, in some embodiments, continuous improvement of the model may be performed by including a “feedback loop” that refines the pre-trained models using responses to feedback on a generated report. When the patient is presented an automatically generated report of his individual situation in electronic, e.g. web-based form, there can be multiple choice options (e.g. “Did you understand the information given in the report [Yes/No]?”; “Do you know what to do with this information? [Yes/No]” etc.) or even a text field for the patient to type questions and comments. The responses can be fed back to the Generative AI module 302 to improve modeling accuracy.

Separately, the report can also be presented as an interactive document, such as a web page. In the interactive document, differences between the actual medical image and the AI-generated healthy version of the same anatomy can be transformed into one another in an animated way, thereby improving the patient's understanding of even subtle differences.

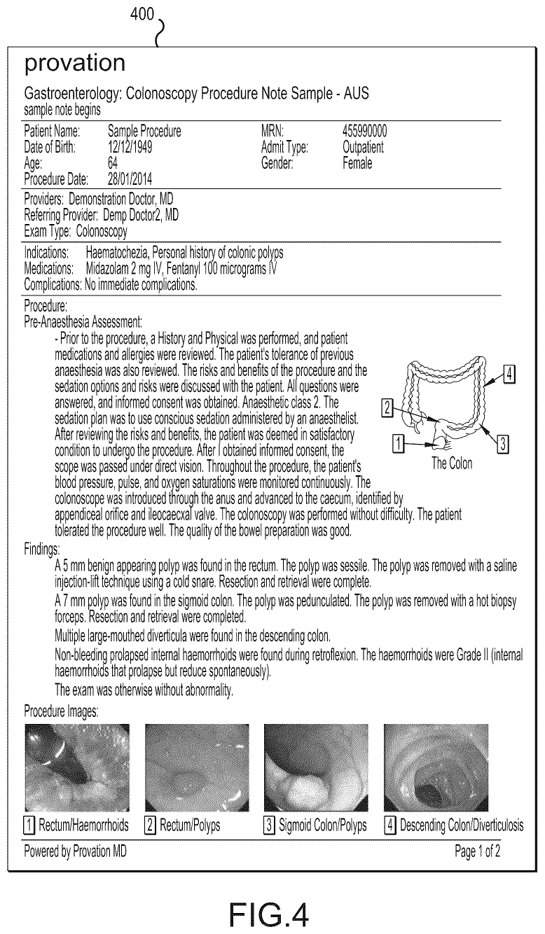

FIGS. 4 and 5 are comparative patient communications for reference in describing an example of a patient-specific communication automatically generated in accordance with embodiments of the inventive concepts.

FIG. 4 illustrates a report in a form and language as used currently for documentation and quality assurance purposes. Often, however, the report is also used as part of the communication with the patient. Unfortunately for the patient, the report is mainly intended for medical staff, and thus the relatively complex medical terms being used are difficult for a patient to understand, as are the implications (e.g. severity of findings, possibly needed follow up, etc.). Also, there is little to no context as to relative severity of the radiological images presented in the report.

FIG. 5 represents an example of a condensed version of the report automatically generated according to the embodiments of the inventive concepts described above. The report is tailored towards the patient's age and education level, and includes a clear indication of relevancy of the findings and recommended course of action. Further, using inpainting as described previously, a simulated healthy state of patient's bowel is depicted in the report to provide additional context.

Embodiments of the inventive concepts include the systems and methods for automatically generating patient-specific reports as described above. In addition, embodiments of the inventive concepts encompass non-transitory computer readable storage media having stored thereon instructions that, when executed by a processor, cause a system carry out the methods of automatically generating patient-specific reports as described above. Examples of such media include random access memory (RAM), read only memory (ROM), optical read/write memory, cache memory, magnetic read/write memory, flash memory, and/or any other non-transitory computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. While representative embodiments are disclosed herein, one of ordinary skill in the art will appreciate that many variations that are in accordance with the present teachings are possible and remain within the scope of the appended claim set. The invention therefore is not to be restricted except within the scope of the appended claims.

What is claimed is:

1. A computer-implemented system for automatically generating patient-specific medical reports, the system comprising:
a processor configured to:
obtain patient-specific information, including patient-specific diagnostic information and communication-related patient-specific meta information;
process the obtained patient-specific information using a pre-trained language model to generate a patient-specific report in a format tailored to the patient's language, education, and expectation level, wherein the pre-trained language model is trained using medical terms and medical data for different age, language, and educational level groups; and
output the generated report.

2. The system of claim 1, wherein the processor is configured to obtain the patient-specific diagnostic information from a variety of sources, including at least one of laboratory results, radiologic findings, or images.

3. The system of claim 2, wherein the processor is further configured to use the collected patient-specific diagnostic information to generate the patient-specific report to include a detailed explanation of the patient's medical condition, potential consequences, and personalized instructions.

4. The system of claim 3, wherein the processor is further configured to generate the patient-specific report to include text with adapted terminology and an extendable full-length detailed report.

5. The system of claim 4, wherein the processor is further configured to create a medical image of the obtained patient-specific diagnostic information showing a radiologic finding and include the medical image in the patient-specific report.

6. The system of claim 5, wherein the processor is further configured to augment a textual explanation provided in the patient-specific report with the medical image.

7. The system of claim 1, wherein the processor is further configured to refine the pre-trained language model using patient feedback responses on the generated patient-specific report.

8. The system of claim 1, further comprising a second processor configured to train the pre-trained language model using the medical terms and medical data for different age, language, and educational level groups.

9. A computer-implemented method for automatically generating patient-specific medical reports, the method comprising:
obtaining patient-specific information, including patient-specific diagnostic information and communication-related patient-specific meta information;
processing the obtained patient-specific information using a pre-trained language model to generate a patient-specific report in a format tailored to the patient's language, education, and expectation level, wherein the pre-trained language model is trained using medical terms and medical data for different age, language, and educational level groups; and
outputting the generated report.

10. The method of claim 9, wherein the patient-specific diagnostic information includes at least one of laboratory results, radiologic findings, or images.

11. The method of claim 10, wherein the patient-specific report is generated to further include a detailed explanation of the patient's medical condition, potential consequences, and personalized instructions.

12. The method of claim 11, wherein the patient-specific report is generated to further include text with adapted terminology and an extendable full-length detailed report.

13. The method of claim 12, further comprising creating a medical image of the obtained patient-specific diagnostic information showing a radiologic finding and include the medical image in the patient-specific report.

14. The method of claim 13, further comprising augmenting a textual explanation provided in the patient-specific report with the medical image.

15. A non-transitory computer-readable medium having stored thereon instructions which, when executed by a processor, cause the processor to:
obtain patient-specific information, including patient-specific diagnostic information and communication-related patient-specific meta information;
process the obtained patient-specific information using a pre-trained language model to generate a patient-specific report in a format tailored to the patient's language, education, and expectation level, wherein the pre-trained language model is trained using medical terms and medical data for different age, language, and educational level groups;
output the generated report.

16. The non-transitory computer-readable medium of claim 15, wherein the patient-specific diagnostic information includes at least one of laboratory results, radiologic findings, or a images.

17. The non-transitory computer-readable medium of claim 16, wherein the instructions, when executed by the processor, further cause the processor to use the patient-specific diagnostic information to generate the patient-specific report to include a detailed explanation of the patient's medical condition, potential consequences, and personalized instructions.

18. The non-transitory computer-readable medium of claim 16, wherein the instructions, when executed by the processor, further cause the processor generate the patient-specific report to include text with adapted terminology and an extendable full-length detailed report.

19. The non-transitory computer-readable medium of claim 18, wherein the instructions, when executed by the processor, further cause the processor create a medical image of the obtained patient-specific diagnostic information showing a radiologic finding and include the medical image in the patient-specific report.

20. The non-transitory computer-readable medium of claim 15, wherein the instructions, when executed by the processor, further cause the processor refine the pre-trained language model using patient feedback responses on the generated patient-specific report.
